(19) United States
(12) Patent Application Publication
(10) Pub. No.: US 2018/0110403 A1
(43) Pub. Date: Apr. 26, 2018

(54) PERICARDIAL ENDOSCOPE SYSTEM

(71) Applicant: OLYMPUS CORPORATION, Tokyo (JP)

(72) Inventors: Yoshiro OKAZAKI, Tokyo (JP); Hiroki HIBINO, Tokyo (JP)

(73) Assignee: OLYMPUS CORPORATION, Tokyo (JP)

(21) Appl. No.: 15/840,133

(22) Filed: Dec. 13, 2017

Related U.S. Application Data
(63) Continuation of application No. PCT/JP2015/067579, filed on Jun. 18, 2015.

(51) Int. Cl. A61B 1/005; A61B 1/01; A61B 1/06; A61B 1/00

(52) U.S. Cl. CPC A61B 1/0051; A61B 1/01; A61B 2017/00243; A61B 1/0008; A61B 1/0661

(57) ABSTRACT

The sheath includes a protruding section that protrudes from a ring-shaped distal end surface of a sheath body and a lifting part that lifts the protruding section within the pericardial cavity to a position separated from the surface of the heart toward the pericardium. The protruding section includes a pressing surface disposed toward the pericardium when the protruding section is lifted by the lifting part, and also includes an opening that is provided at a position facing the pressing surface in a radial direction and at which a distal end surface of the endoscope is capable of appearing in the radial direction as an endoscope bending section operates.

### CROSS-REFERENCE TO RELATED APPLICATIONS

This is a continuation of International Application PCT/JP2015/067579, with an international filing date of Jun. 18, 2015, which is hereby incorporated by reference herein in its entirety.

### TECHNICAL FIELD

The present invention relates to pericardial endoscope systems.

### BACKGROUND ART

A known cardiac device in the related art has a bending section at the distal end thereof and is used by being percutaneously inserted into the pericardium from under the xiphoid process (e.g., see Non Patent Literature 1 and Patent Literature 1). With regard to a catheter for cardiac ablation in Non Patent Literature 1, the bending section is bendable in the direction parallel to the surface of the heart. An endoscope in Patent Literature 1 is for setting a lead wire from the interior of the pericardium to the exterior of the pericardium. By bending the bending section toward the pericardium to bring the distal end surface thereof into contact with the pericardium, and then causing the lead wire to protrude from the distal end surface, the lead wire can be pierced through the pericardium.

### CITATION LIST

### Non Patent Literature

{NPL 1}

Barbara Natterson Horowitz, et al. “Percutaneous intrapericardial echocardiography during catheter ablation: a feasibility study” Heart Rhythm, 2006, Volume 3, Issue 11, pp. 1275-1282

### Patent Literature

{PTL 1}

Japanese Unexamined Patent Application, Publication No. 2010-29564

### SUMMARY OF INVENTION

A first aspect of the present invention is directed to a pericardial endoscope sheath that is narrow and cylindrical and that is insertable into a pericardial cavity between a heart and a pericardium, the pericardial endoscope sheath including: a flexible cylindrical sheath body that is bendable in conformity to a shape of a surface of the heart within the pericardial cavity and that has openings at opposite ends, a protruding section that protrudes in a longitudinal direction of the sheath body from a ring-shaped distal end surface of the sheath body, and a lifting part that generates a force for lifting the protruding section disposed within the pericardial cavity to a position separated from the surface of the heart toward the pericardium against an elastic force of the pericardium, wherein the protruding section includes a pressing surface that is provided in a circumferential portion centered on a longitudinal axis of the sheath body and that is disposed toward the pericardium when the protruding section is lifted by the lifting part so as to press against the pericardium, and an opening that is provided at a position facing the pressing surface in a radial direction and at which a distal end surface of the endoscope protruding to the protruding section from the opening in the distal end surface of the sheath body is capable of appearing and disappearing in the radial direction as the endoscope bending section operates, in a state in which the endoscope is inserted into the sheath in a movable manner in a longitudinal direction

A second aspect of the present invention is directed to a pericardial endoscope system comprising: the pericardial endoscope sheath according to claim 1; and an endoscope that is inserted into the pericardial endoscope sheath in a movable manner in a longitudinal direction and that is provided with an endoscope bending section at a distal end.

### BRIEF DESCRIPTION OF DRAWINGS

FIG. 1 illustrates the overall configuration of a pericardial endoscope system according to an embodiment of the present invention.

FIG. 2 illustrates a state where the pericardial endoscope system in FIG. 1 is being used.

FIG. 3 is a cross-sectional view of the pericardial endoscope system in FIG. 2, taken along line II-II.

FIG. 4 is a perspective view illustrating a distal end section of a sheath in the pericardial endoscope system in FIG. 1.

FIG. 5 is a side view of the sheath and an endoscope, as viewed from an opening side, when the endoscope is inserted into the sheath by a predetermined amount.

FIG. 6 illustrates a third marker provided on the sheath and a fourth marker provided on the endoscope.

FIG. 7 illustrates a dilator used when inserting the sheath in FIG. 1 into the pericardium.

FIG. 8 illustrates the operation of a sheath bending section and a protruding section in the pericardial cavity.

FIG. 9 is a cross-sectional view illustrating a modification of the protruding section.

FIG. 10 is a cross-sectional view illustrating another modification of the protruding section.

FIG. 11 illustrates another modification of the protruding section.

FIG. 12 illustrates another modification of the protruding section.

FIG. 13 illustrates another modification of the protruding section.

FIG. 14 illustrates another modification of the protruding section.

FIG. 15 illustrates another modification of the protruding section.

FIG. 16 illustrates another modification of the protruding section.

FIG. 17 illustrates another modification of the protruding section.

FIG. 18 illustrates another modification of the protruding section.

FIG. 19 illustrates another modification of the protruding section.

FIG. 20 is a front view of the protruding section in FIG. 19, as viewed from the distal end.

FIG. 21 illustrates a modification of the sheath.

FIG. 22 illustrates another modification of the sheath.

### DESCRIPTION OF EMBODIMENTS

A pericardial endoscope system 100 according to an embodiment of the present invention will be described below with reference to the drawings.

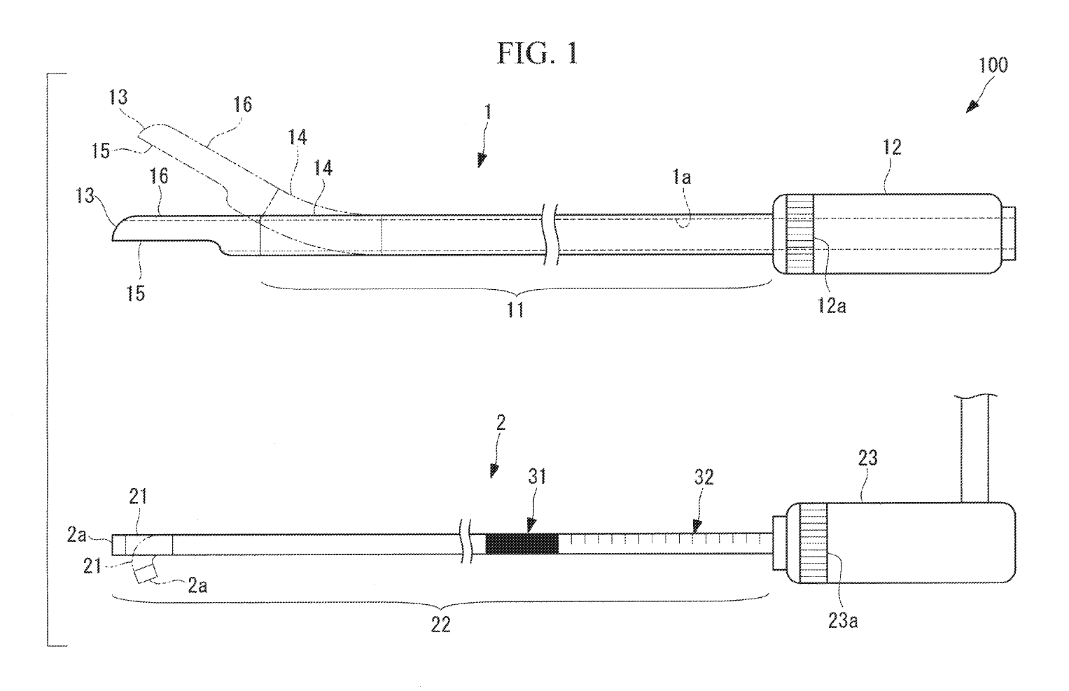

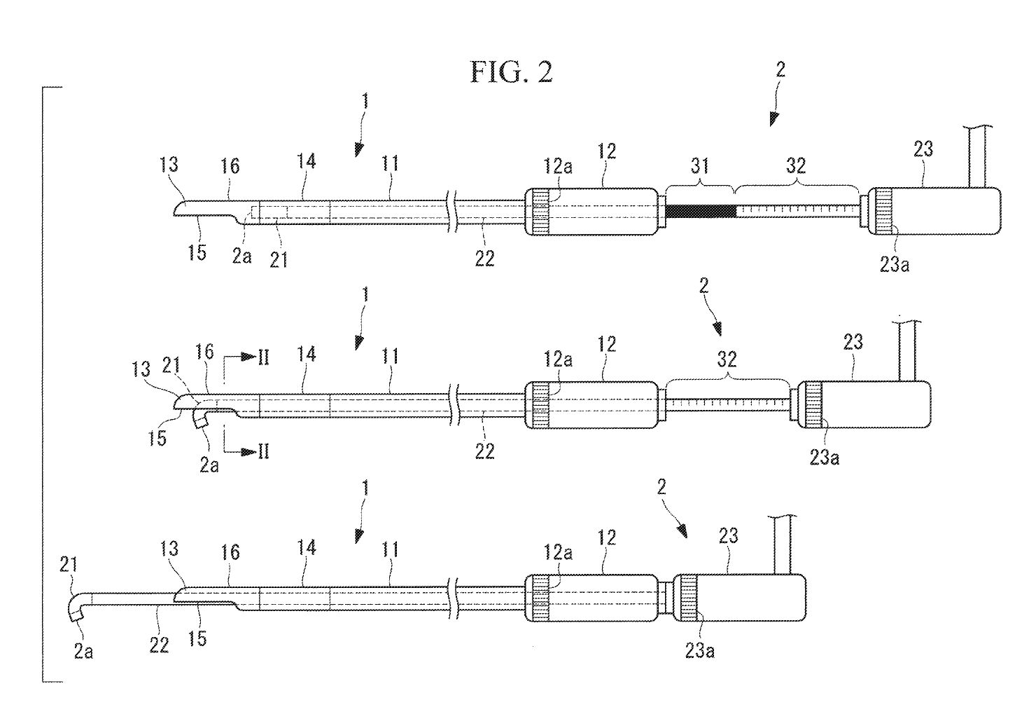

As shown in FIGS. 1 and 2, the pericardial endoscope system 100 according to this embodiment includes a sheath 1 to be percutaneously inserted into the pericardium and an endoscope 2 to be inserted into the sheath 1.

The sheath 1 includes a narrow cylindrical sheath body 11 having openings at opposite ends thereof, an operating section 12 connected to the base end of the sheath body 11, and a protruding section 13 connected to the distal end of the sheath body 11. The sheath 1 has a through-hole 1a extending longitudinally therethrough from the distal end surface of the protruding section 13 to the base end surface of the operating section 12 and having an inner diameter larger than the outer diameter of an insertion section 22 (to be described later) of the endoscope 2. Accordingly, as shown in the upper part, the middle part, and the lower part of FIG. 2, the operating section 22 of the endoscope 2 inserted in the sheath 1 can be moved in the longitudinal direction. The sheath body 11 and the protruding section 13 are composed of an X-ray impermeable material so that the sheath body 11 and the protruding section 13 inserted in the body can be observed from outside the body by using a fluoroscopic device.

The sheath body 11 has flexibility such that the sheath body 11 is bendable in conformity to the shape of the surface of tissue within a biological organism when the sheath body 11 comes into contact with the tissue. The distal end of the sheath body 11 is provided with a sheath bending section (i.e., a lifting part) 14 that is bendable.

The operating section 12 includes a dial-type bend control section 12a for controlling the bending angle of the sheath bending section 14. The bend control section 12a and the distal end of the sheath bending section 14 are connected to each other by means of a bending wire (not shown) that extends longitudinally through the interior of the sidewall of the sheath body 11. By being rotated by an operator, the bend control section 12a pulls the bending wire toward the base end by a distance corresponding to the rotated amount so as to bend the sheath bending section 14 by a bending angle corresponding to the rotated amount.

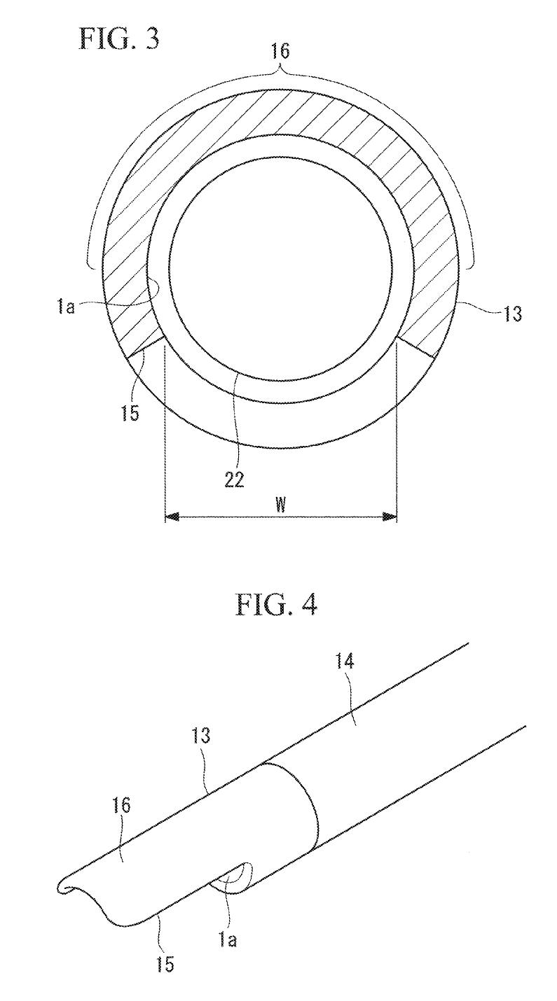

As shown in FIGS. 3 and 4, the protruding section 13 is a cylindrical member that protrudes in the longitudinal direction of the sheath body 11 from the ring-shaped distal end surface of the sheath body 11 and has openings at opposite ends thereof. The protruding section 13 has an opening 15 formed by longitudinally cutting a circumferential portion thereof from the distal end thereof to an intermediate position in the longitudinal direction.

In the outer peripheral surface of the protruding section 13, a circumferential portion that faces the opening 15 in the radial direction serves as a pressing surface 16 that presses against a pericardium B by coming into contact with the pericardium B when the pericardium B is lifted as a result of the bending operation of the sheath bending section 14. This will be described later. Therefore, the protruding section 13 has enough rigidity for maintaining its shape against the elastic force of the pericardium B when the pericardium B is lifted. For example, the protruding section 13 is composed of an elastic resin material, such as polyurethane rubber. As indicated by a two-dot chain line in FIG. 1, the sheath bending section 14 is bendable toward the same side as the pressing surface 16. An edge formed by the distal end surface and the outer peripheral surface of the protruding section 13 is round-chamfered.

The endoscope 2 is a direct-view-type flexible endoscope used for observing the visual field in front of a distal end surface 2a. The endoscope 2 includes a narrow insertion section 22 provided with an endoscope bending section 21 at the distal end thereof, and also includes an operating section 23 connected to the base end of the insertion section 22. An endoscopic image acquired by the endoscope 2 is displayed on a display unit (not shown).

A minimum width W of the opening 15 in the radial direction of the sheath body 11 is slightly larger than the outer diameter of the endoscope bending section 21. Accordingly, as shown in the middle part of FIG. 2, when the endoscope bending section 21 bends toward the opening 15 within the protruding section 13, the distal end surface 2a of the endoscope 2 can protrude in the radial direction from the opening 15. Furthermore, in the state where the distal end surface 2a of the endoscope 2 protrudes from the opening 15, the endoscope bending section 21 is stopped from rotating about the longitudinal axis of the insertion section 22 by the circumferential end surfaces of the opening 15. Accordingly, the distal end surface 2a of the endoscope 2 does not swivel about the longitudinal axis of the insertion section 22.

The operating section 23 includes a dial-type bend control section 23a for controlling the bending angle of the endoscope bending section 21. The bend control section 23a has a configuration similar to that of the bend control section 12a of the sheath 1. Specifically, the bend control section 23a is connected to the distal end of the endoscope bending section 21 by means of a bending wire (not shown) that extends longitudinally through the insertion section 22, and can bend the endoscope bending section 21 by a bending angle corresponding to the rotated amount of the bend control section 23a.

As shown in FIG. 2, the outer peripheral surface of the base end of the insertion section 22 is provided with a first marker (marker) 31 and a second marker (marker) 32 indicating an amount by which the insertion section 22 is inserted in the sheath 1. The second marker 32 is provided toward the base end relative to the first marker 31. The first marker 31 is provided uniformly on the outer peripheral surface of the insertion section 22 by, for example, painting the outer peripheral surface of the insertion section 22 in a single color. The second marker 32 is a scale and has a plurality of lines provided at equal intervals (e.g., 5 mm intervals) in the longitudinal direction. The color of the first marker 31 and the color of the second marker 32 are different from each other so that the operator can easily recognize the boundary between the first marker 31 and the second marker 32.

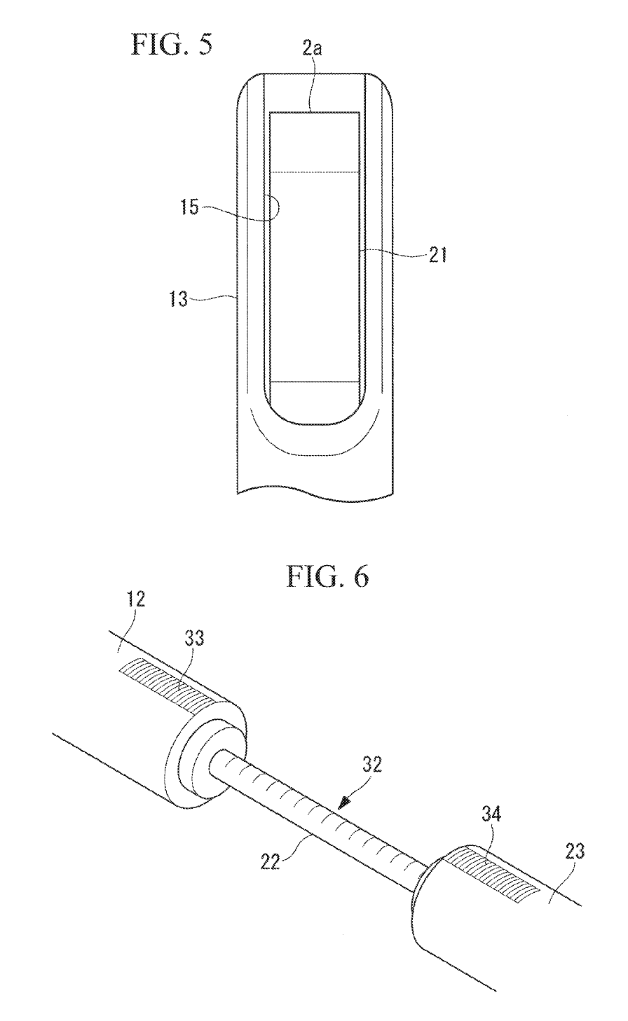

The position of the boundary between the first marker 31 and the second marker 32 in the longitudinal direction of the insertion section 22 is aligned with the base end of the operating section 12 of the sheath 1 when the insertion section 22 is inserted into the sheath 1 by a predetermined amount. As shown in FIG. 5, the predetermined amount is an amount by which the endoscope bending section 21 is disposed at the same position as the opening 15 in the longitudinal direction. When the insertion section 22 is inserted into the sheath 1 by the predetermined amount, the endoscope bending section 21 is bent toward the opening 15 so that the distal end surface 2a of the endoscope 2 can protrude from the opening 15.

As shown in FIG. 6, the operating section 12 of the sheath 1 and the operating section 23 of the endoscope 2 are provided in circumferential portions of respective outer peripheral surfaces and are provided with a third marker 33 and a fourth marker 34, respectively, indicating a relative rotational angle between the sheath 1 and the endoscope 2 about the longitudinal axis. The third marker 33 and the fourth marker 34 are provided such that the rotational angles of the two markers 33 and 34 about the longitudinal axis match and such that the two markers 33 and 34 are arranged in a single line in the longitudinal direction when the rotational angle of the insertion section 22 about the longitudinal axis within the sheath 1 is an angle that allows the endoscope bending section 21 to bend at the opening 15 side.

Next, the operation of the pericardial endoscope system 100 having the above-described configuration will be described.

In order to observe the heart A by using the pericardial endoscope system 100 according to this embodiment, the sheath body 11 is first percutaneously inserted into the pericardium from under the xiphoid process.

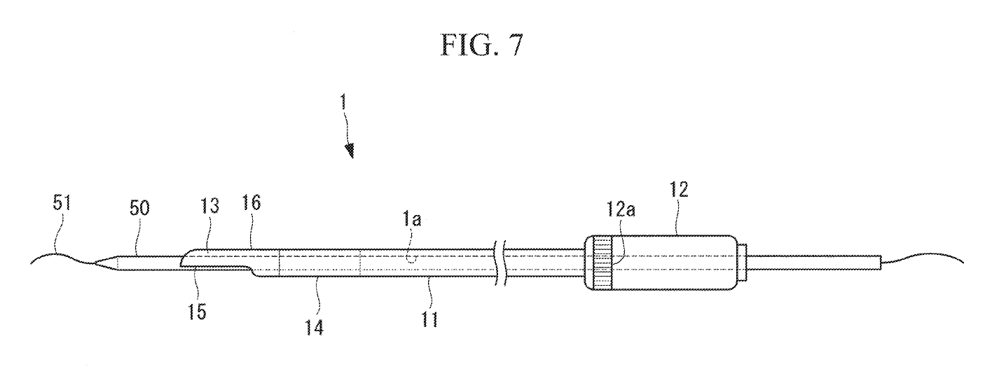

For the insertion of the sheath body 11, a dilator 50 shown in FIG. 7 is used. The dilator 50 is a narrow member that can be inserted longitudinally into the through-hole 1a of the sheath 1 and has a substantially conical distal end section whose diameter gradually decreases toward the distal end. The dilator 50 also has a hole for a guide wire 51 that extends therethrough in the longitudinal direction.

The sheath 1 is attached to the dilator 50 by inserting the dilator 50 into the sheath 1 such that the distal end section of the dilator 50 protrudes further than the protruding section 13, and the dilator 50 and the sheath 1 are moved forward together along the guide wire 51 in a state where the guide wire 51 extends through the dilator 50. The guide wire 51 is preliminarily extended to a desired observation site within the pericardial cavity C from under the xiphoid process by using, for example, a puncture needle (not shown).

Because the distal end section of the dilator 50 has a substantially conical shape that gradually becomes thicker from the distal end toward the base end, the protruding section 13 and the sheath body 11 together with the dilator 50 can be readily inserted into the pericardial cavity C while using the distal end section of the dilator 50 to gradually increase the diameter of a small hole in the pericardium B through which the guide wire 51 extends. After the sheath body 11 is inserted to the observation site within the pericardial cavity C, the guide wire 51 and the dilator 50 are pulled out while the sheath body 11 is left remaining in the pericardial cavity C.

Subsequently, the rotational angle of the sheath body 11 about the longitudinal axis is adjusted so that the pressing surface 16 faces the pericardium B. Then, the bend control section 12a of the sheath 1 is controlled so as to cause the sheath bending section 14 to bend toward the pericardium B. Thus, the protruding section 13 disposed on the surface of the heart A is lifted toward the pericardium B, and the pressing surface 16 in contact with the pericardium B presses the pericardium B outward.

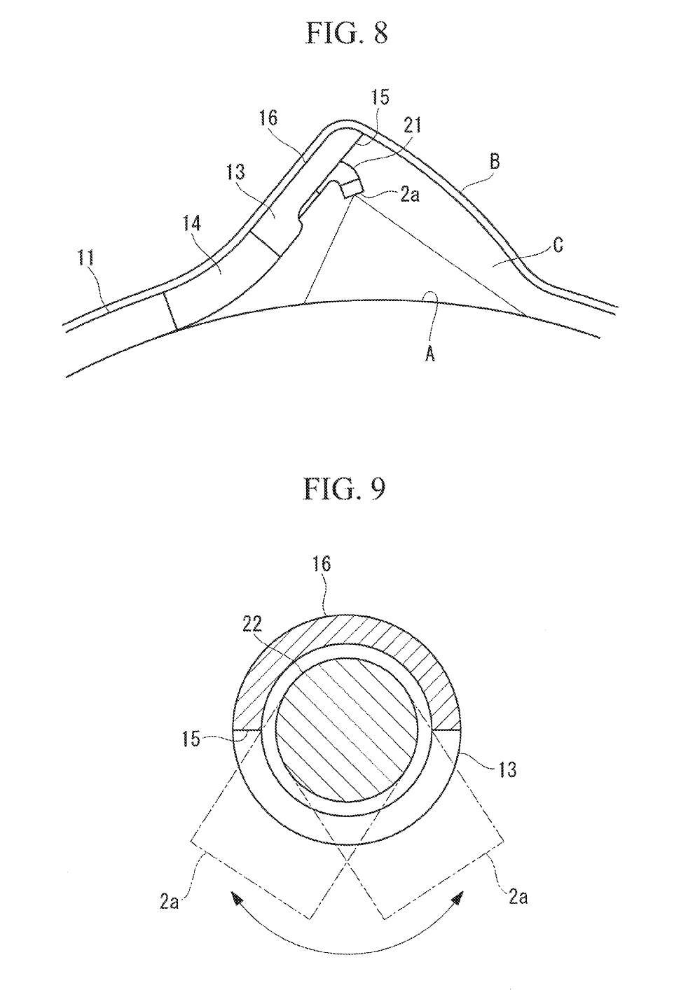

In this case, a pressing force acts inward (i.e., toward the heart A) on the pressing surface 16 from the pericardium B having elasticity. However, because the sheath bending section 14 bent by the bend control section 12a has rigidity high enough for maintaining its bent shape against the elastic force of the pericardium B, the protruding section 13 moves away from the surface of the heart A while lifting the pericardium B as the bending angle of the sheath bending section 14 increases, as shown in FIG. 8. A space is ensured between the heart A and the protruding section 13 lifted together with the pericardium B. In this state, the opening 15 faces the surface of the heart A. The above operation of the sheath 1 inside the body is performed while the sheath 1 is observed using a fluoroscopic device.

Subsequently, the insertion section 22 of the endoscope 2 is inserted into the through-hole 1a from the base end of the sheath 1, and the insertion section 22 is moved forward until the boundary between the first marker 31 and the second marker 32 added to the outer peripheral surface of the insertion section 22 is positioned at the base end of the sheath 1. Thus, the insertion section 22 is inserted into the sheath body 11 by the predetermined amount, so that the endoscope bending section 21 can be disposed at the same position as the opening 15 in the longitudinal direction.

Then, the insertion section 22 is rotated about the longitudinal axis within the sheath 1 while the angle of the sheath 1 about the longitudinal axis is maintained such that the markers 33 and 34 respectively provided on the operating sections 12 and 23 are arranged in a single line. Thus, the angle of the insertion section 22 about the longitudinal axis is adjusted such that the endoscope bending section 21 is bendable at the opening 15 side, that is, at the heart A side.

Subsequently, the endoscope bending section 21 is bent by operating the bend control section 23a of the endoscope 2. Consequently, the endoscope bending section 21 bends toward the heart A so that the distal end surface 2a of the endoscope 2 protrudes in the radial direction from the opening 15 facing the surface of the heart A. Because the distal end surface 2a of the endoscope 2 protruding from the opening 15 faces the surface of the heart A at a position separated from the surface of the heart A by an observation distance, a bird's eye view of a wide region of the surface of the heart A can be observed from the front side of the region by using the endoscope 2. Since the distal end surface 2a is stopped from swiveling by means of the end surfaces of the opening 15 in this state, the position of the visual field of the endoscope 2 can be made stable even when body motions caused by heartbeats and breathing occur.

According to this embodiment, the protruding section 13 provided at the distal end of the sheath body 11 is lifted from the surface of the heart A against the elastic force of the pericardium B by bending the sheath bending section 14, so that an observation distance can be ensured between the protruding section 13 and the surface of the heart A within the pericardial cavity C. Furthermore, because the opening 15 is disposed at the heart A side in the state where the protruding section 13 is lifted, the distal end surface 2a of the endoscope 2 can be disposed facing the surface of the heart A by bending the endoscope bending section 21. This is advantageous in that a bird's eye view of a wide region of the surface of the heart A can be observed by using the endoscope 2. Moreover, this is also advantageous in that the distance between the distal end surface 2a of the endoscope 2 and the surface of the heart A can be readily controlled by adjusting the bending angle of the sheath bending section 14.

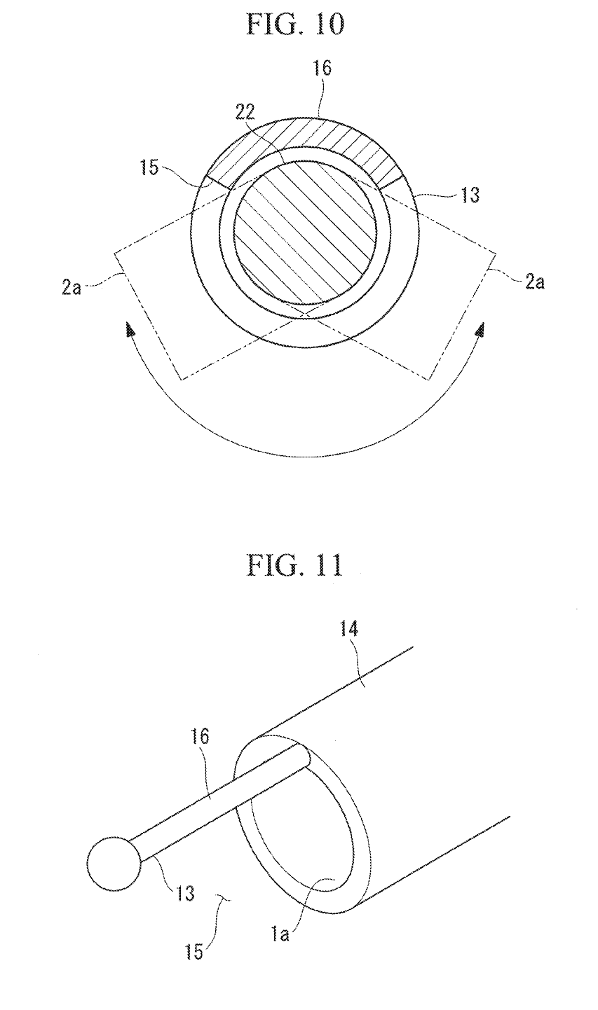

As shown in FIGS. 9 and 10, in this embodiment, the opening 15 may have a larger width such that the distal end surface 2a of the endoscope 2 protruding from the opening 15 is swivelable. The opening 15 in FIG. 9 is formed by cutting out the protruding section 13 at 180°. The opening 15 in FIG. 10 is formed by cutting out the protruding section 13 at an angle larger than 180°. Accordingly, by swiveling the distal end surface 2a of the endoscope 2 to move the visual field of the endoscope 2 in the circumferential direction of the protruding section 13, a wider region can be observed.

Although the protruding section 13 has a cylindrical shape obtained by cutting out a circumferential portion thereof in this embodiment, the shape of the protruding section 13 is not limited to this shape and may be changed, where appropriate. FIGS. 11 to 13 illustrate modifications of the protruding section 13.

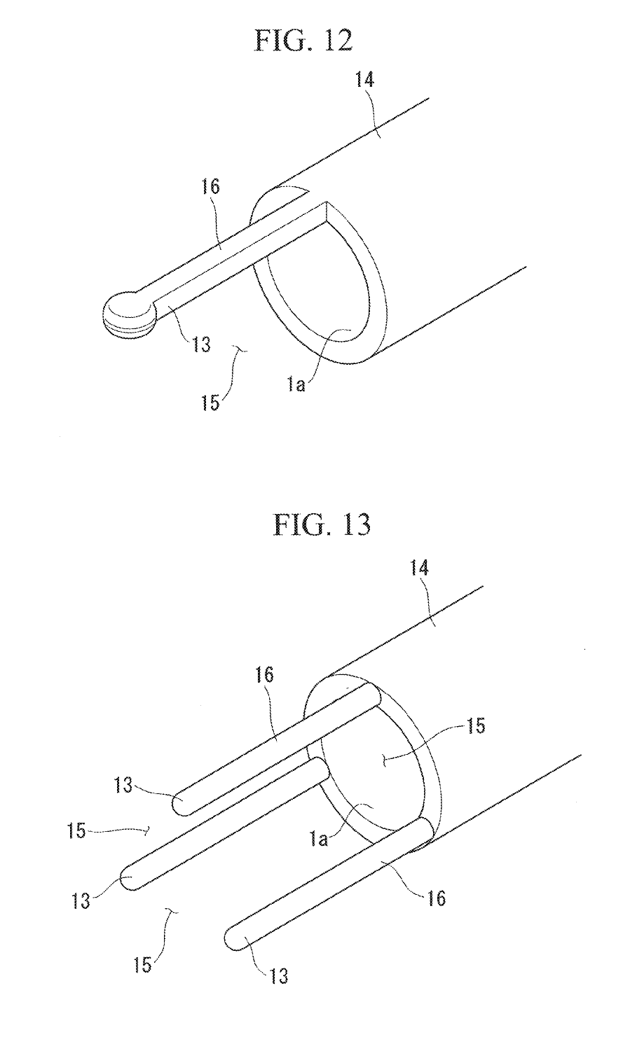

As shown in FIGS. 11 to 13, the protruding section 13 may be a rod-shaped member that protrudes from the distal end surface of the sheath body 11. The distal end of the rod-shaped protruding section 13 preferably has a round shape. With regard to such a protruding section 13, only a single section may be provided, as shown in FIGS. 11 and 12, or a plurality of sections may be provided at intervals in the circumferential direction of the sheath body 11, as shown in FIG. 13 (i.e., three sections in the example shown in the drawing). With regard to the rod-shaped protruding section 13, the radially outer surface thereof serves as the pressing surface 16. Furthermore, with regard to the protruding sections 13 in FIG. 13, a gap between two neighboring protruding sections 13 in the circumferential direction serves as the opening 15.

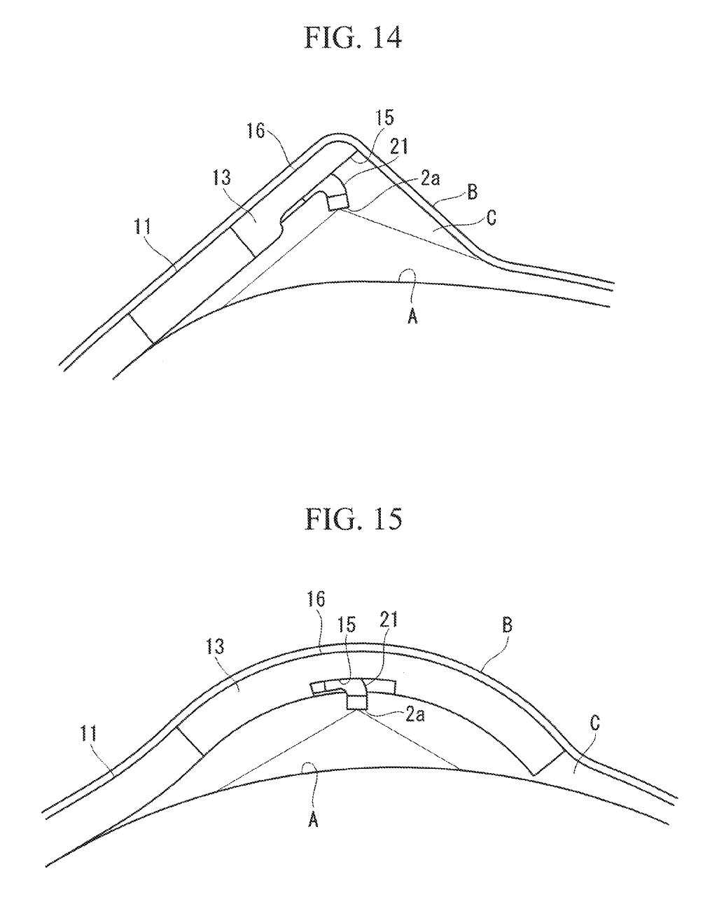

Furthermore, as shown in FIGS. 14 and 15, in this embodiment, the sheath body 11 may include, in place of the sheath bending section 14, a protruding section 13 having high rigidity as a lifting part that can maintain a straight shape or a bent shape having a curvature smaller than the curvature in the natural state of the pericardium B against the elastic force of the pericardium B within the pericardial cavity C. Accordingly, the protruding section 13 can be lifted from the surface of the heart A together with the pericardium B within the pericardial cavity C by utilizing the rigidity of the protruding section 13. Moreover, the bending wire for bending the sheath bending section 14 can be omitted so that the sheath body 11 can be reduced in diameter.

In the protruding section 13 shown in FIGS. 14 and 15, the opening 15 is provided at a position where the distance from the surface of the heart A is substantially at a maximum. The protruding section 13 in FIG. 14 is substantially straight in the longitudinal direction of the sheath body 11 and has enough rigidity to maintain its straight shape within the pericardial cavity C. Therefore, the opening 15 is preferably provided at the distal end of the protruding section 13.

The protruding section 13 in FIG. 15 has enough rigidity to bend at a curvature smaller than the curvature of the surface of the heart A against the elastic force of the pericardium B within the pericardial cavity C, and the opening 15 is provided at an intermediate position of the protruding section 13 in the longitudinal direction. According to the protruding section 13 in FIG. 15, a space can be ensured between the pericardium B and the surface of the heart A at the distal end relative to the opening 15 by means of the pressing surface 16 located toward the distal end relative to the opening 15. Therefore, by bending the endoscope bending section 21 by a small bending angle, the surface of the heart A at the distal end can also be observed.

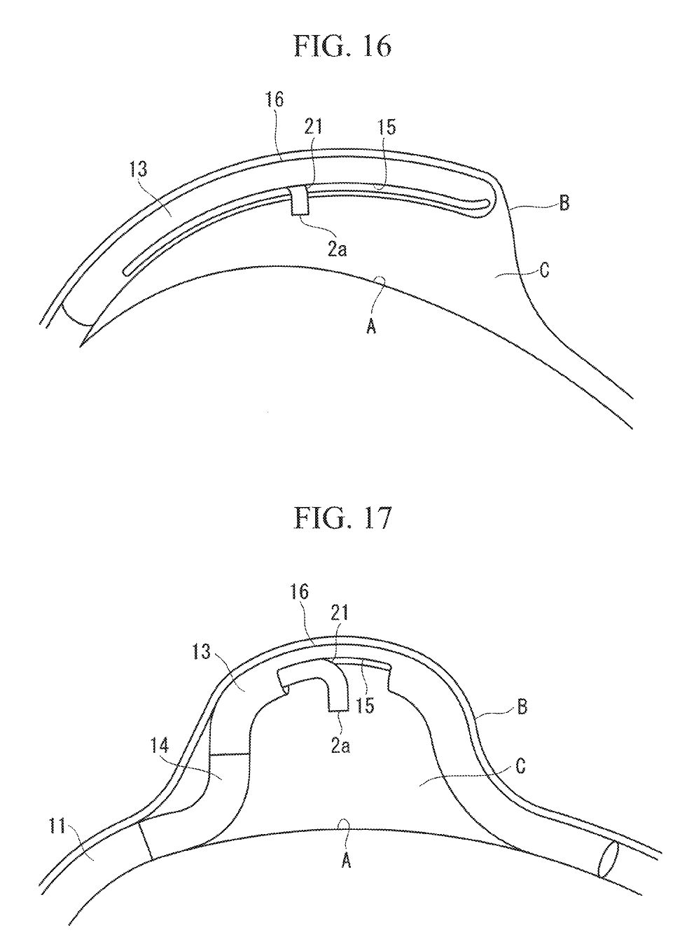

With regard to the high-rigidity protruding section 13 that bends at a curvature smaller than the curvature of the surface of the heart A within the pericardial cavity C, as in FIG. 15, an opening 15 that extends long in the longitudinal direction from the distal end toward the base end of the protruding section 13 may be provided, as shown in FIG. 16. With the protruding section 13 in FIG. 16, the distal end surface 2a of the endoscope 2 protruding from the opening 15 can also be moved in the longitudinal direction of the sheath body 11 by moving the insertion section 22 in the longitudinal direction within the sheath body 11, whereby the visual field can be moved.

Furthermore, the protruding section 13 provided with the opening 15 at the intermediate position in the longitudinal direction may be applied to the sheath body 11 equipped with the sheath bending section 14, as shown in FIG. 17. In this case, since the protruding section 13 can be lifted toward the pericardium B by bending the sheath bending section 14, the protruding section 13 does not need high rigidity. Accordingly, a space can be ensured between the pericardium B and the surface of the heart A toward the distal end relative to the opening 15 by means of the pressing surface 16 located toward the distal end relative to the opening 15. Moreover, in the case there the protruding section 13 having high rigidity in FIGS. 14 to 16 is used, it is difficult to access, for example, the back side of the heart A, and the observation site is thus limited. In contrast, with the protruding section 13 having high flexibility in FIG. 17, a wider region within the pericardial cavity C can be accessed.

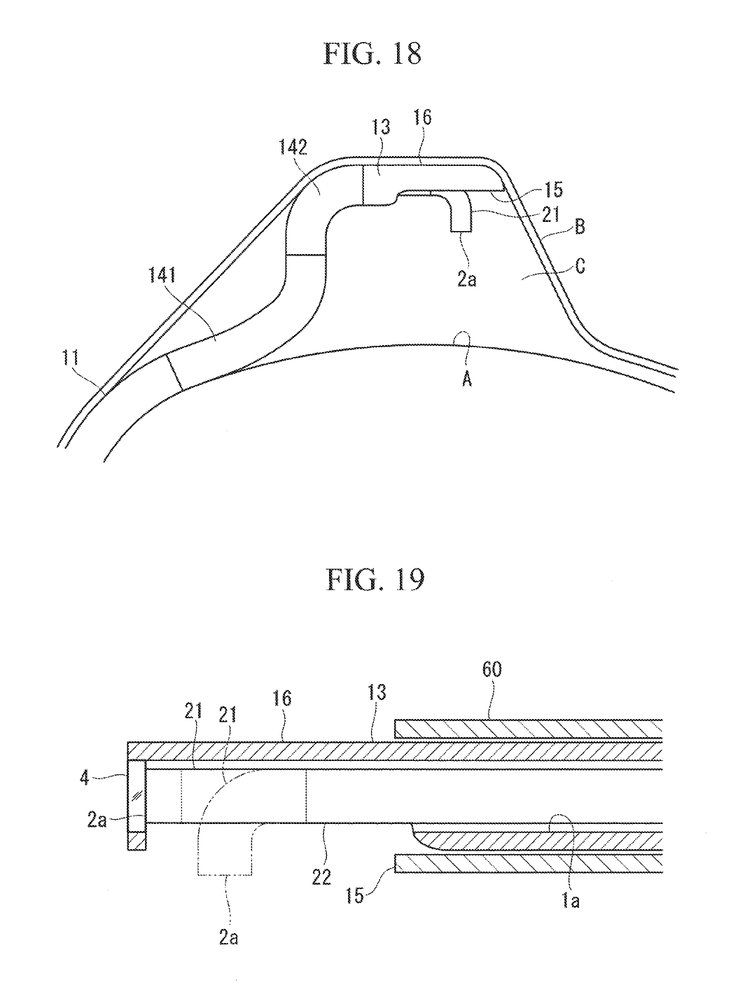

Furthermore, as shown in FIG. 18, in this embodiment, the sheath body 11 may include two sheath bending sections 141 and 142 that are coupled to each other in the longitudinal direction.

The first sheath bending section 141 at the base end is bendable toward the pressing surface 16, similar to the sheath bending section 14. The second sheath bending section 142 at the distal end is bendable toward the opposite side from the first sheath bending section 141, that is, toward the opening 15.

According to the sheath body 11 in FIG. 18, the protruding section 13 can be lifted toward the pericardium B by bending the first sheath bending section 141, and the lifting amount of the protruding section 13 can be finely adjusted by changing the bending angle of the second sheath bending section 142. This is advantageous in that the observation distance can be controlled with more accuracy. The first sheath bending section 141 and the second sheath bending section 142 may be bent in cooperation with each other by operating a shared bend control section 12a, or may be bent independently from each other by operating two bend control sections 12a provided in correspondence with the two respective sheath bending sections.

Furthermore, as shown in FIG. 19, in this embodiment, the distal end surface of the protruding section 13 may be provided with a transparent plate 4 that is at least transparent with respect to visible light.

Accordingly, when the endoscope bending section 21 is extended straight within the protruding section 13, the visual field in front of the distal end of the protruding section 13 can be observed via the transparent plate 4. Therefore, the inserting operation of the sheath body 11 can be performed while observing the forward visual field in the inserting direction by bringing the distal end surface 2a of the endoscope 2 into abutment with the transparent plate 4.

In this case, the opening 15 is provided at the distal end of the protruding section 13 so that the distal end surface 2a of the endoscope 2 can protrude from the opening 15 by bending the endoscope bending section 21 from the state where the distal end surface 2a is butted against the transparent plate 4. Therefore, the operator may simply insert the insertion section 22 into the sheath 1 to a position where the distal end surface 2a abuts on the transparent plate 4, whereby the insertion section 22 can be inserted by a predetermined amount by which the endoscope bending section 21 is disposed at the same position as the opening 15 in the longitudinal direction.

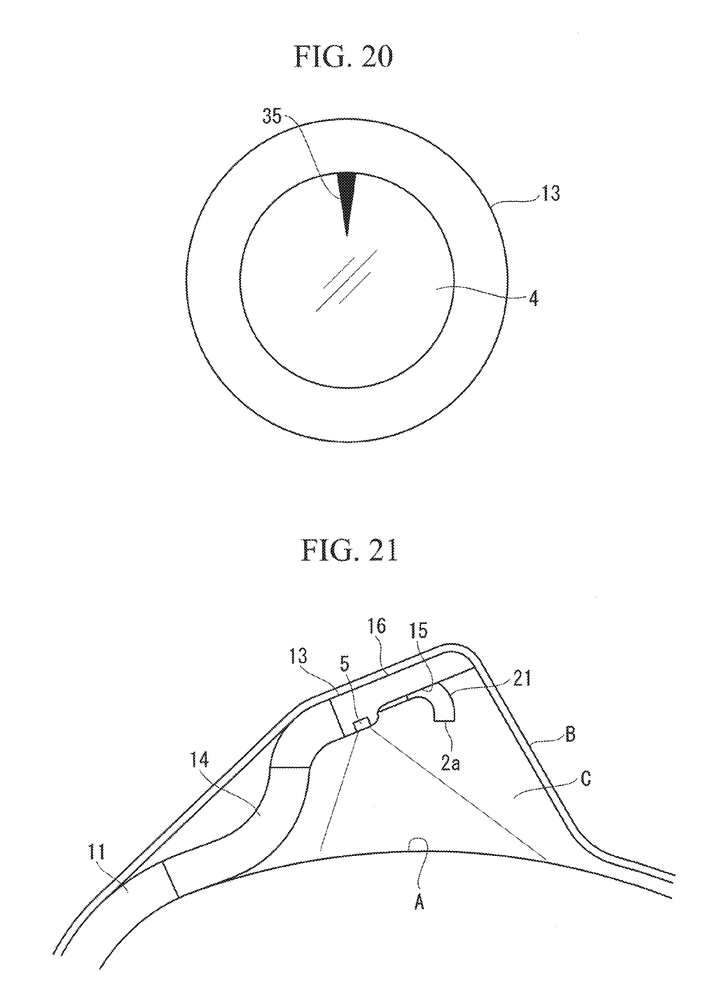

As shown in FIG. 20, the transparent plate 4 may be provided with a marker 35 that is located at a predetermined position in the circumferential direction (e.g., at a position at the pressing surface 16 side) and that indicates the angle of the sheath body 11 about the longitudinal axis. Accordingly, the operator may observe the position of the marker 35 within an endoscopic image so as to readily recognize the relative rotational angle between the sheath body 11 and the insertion section 22. Thus, without having to check the markers 33 and 34 on the operating sections 12 and 23, the rotational angle of the insertion section 22 can be adjusted such that the endoscope bending section 21 is bendable at the opening 15 side.

In the sheath 1 in FIG. 20, the through-hole 1a is blocked by the transparent plate 4 at the distal end of the protruding section 13, meaning that the dilator 50 shown in FIG. 7 cannot be used when the sheath 1 is inserted in the pericardium. Therefore, another sheath 60 having a diameter larger than that of the sheath body 11 may be inserted into the pericardium by using the dilator 50, and the sheath body 11 may be inserted into the pericardium along a route ensured inside the sheath 60.

Furthermore, as shown in FIG. 21, in this embodiment, the sheath 1 may include an illuminating section 5 that is provided in the protruding section 13 near the opening 15 and that outputs illumination light outward in the radial direction of the opening 15. The position of the illuminating section 5 is not particularly limited so long as it is located where it can effectively illuminate a radially outer region of the opening 15. For example, the illuminating section 5 may be provided in the outer peripheral surface at the base end of the opening 15.

The illuminating section 5 is, for example, an optical fiber whose output end surface is fixed near the opening 15 and that extends internally through the sidewall of the sheath body 11 so as to be connected to a light source disposed at the base end of the sheath 1. The illuminating section 5 may be a light source, such as an LED, or may be a light emitting member or a fluorescent member.

According to the sheath 1 in FIG. 21, the surface of the heart A facing the opening 15 can be illuminated with the illumination light output from the illuminating section 5. Because the space for an illumination member (e.g., an illumination lens or an optical fiber) that can be accommodated within the narrow insertion section 22 is limited, for example, only a small number of optical fibers can be accommodated in the insertion section 22, resulting in a reduced amount of illumination light. According to this modification, the sheath 1 is provided with an illuminating function so as to compensate for the amount of illumination light for illuminating the visual field of the endoscope 2. Alternatively, the illumination member within the insertion section 22 may be omitted so that the diameter of the insertion section 22 can be reduced.

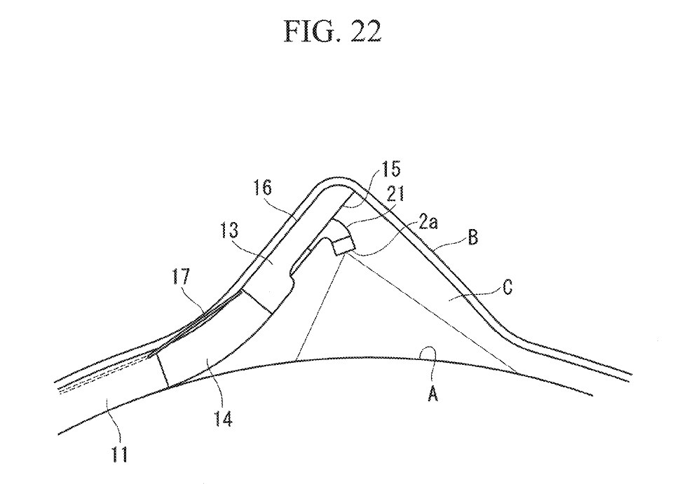

As an alternative to this embodiment in which a bending wire 17 of the sheath 1 extends internally through the entire length of the sheath body 11, the bending wire 17 may be disposed in the sheath bending section 14 so as to be exposed to the outer side of the sheath body 11, as shown in FIG. 22.

In the sheath body 11, the bending wire 17 comes into contact with surrounding components, resulting in a loss of pulling force applied to the bending wire 17 from the bend control section 12a. By disposing the distal end of the bending wire 17 at the outer side of the sheath bending section 14, a loss in the pulling force is reduced, so that a larger pulling force is transmitted to the distal end of the sheath bending section 14. Consequently, the bent shape of the sheath bending section 14 is made more stable, whereby the protruding section 13 and the pericardium B can be lifted more stably.

Furthermore, as an alternative to this embodiment in which the endoscope 2 is of a direct viewing type, the endoscope 2 may be of an oblique viewing type or a side viewing type. In the case where an oblique viewing endoscope is used, the bending angle of the endoscope bending section 21 when observing the surface of the heart A can be small, so that a larger observation distance can be ensured. In the case where a side viewing endoscope is used, the endoscope bending section 21 does not have to be bent when observing the surface of the heart A, so that an even larger observation distance can be ensured.

As a result, the above-described embodiment leads to the following aspects.

A first aspect of the present invention is directed to a pericardial endoscope sheath that is narrow and cylindrical and that is insertable into a pericardial cavity between a heart and a pericardium, the pericardial endoscope sheath including: a flexible cylindrical sheath body that is bendable in conformity to a shape of a surface of the heart within the pericardial cavity and that has openings at opposite ends, a protruding section that protrudes in a longitudinal direction of the sheath body from a ring-shaped distal end surface of the sheath body, and a lifting part that generates a force for lifting the protruding section disposed within the pericardial cavity to a position separated from the surface of the heart toward the pericardium against an elastic force of the pericardium, wherein the protruding section includes a pressing surface that is provided in a circumferential portion centered on a longitudinal axis of the sheath body and that is disposed toward the pericardium when the protruding section is lifted by the lifting part so as to press against the pericardium, and an opening that is provided at a position facing the pressing surface in a radial direction and at which a distal end surface of the endoscope protruding to the protruding section from the opening in the distal end surface of the sheath body is capable of appearing and disappearing in the radial direction as the endoscope bending section operates, in a state in which the endoscope is inserted into the sheath in a movable manner in a longitudinal direction

With the pericardial endoscope sheath according to this aspect, the sheath body is percutaneously inserted into the pericardial cavity from outside the body, the endoscope is subsequently inserted into the sheath body, and then the distal end surface of the endoscope is caused to protrude to the protruding section from the opening in the distal end surface of the sheath body. Thus, the interior of the pericardial cavity can be observed with the endoscope through the opening in the protruding section.

In this case, the protruding section disposed such that the pressing surface is positioned at the pericardium side is lifted by the lifting part toward the pericardium relative to the surface of the heart, so that a space is ensured between the protruding section and the surface of the heart. Because the opening is disposed at the heart side in this state, the distal end surface of the endoscope is caused to protrude toward the heart through the opening by bending the endoscope bending section disposed at the protruding section toward the heart, so that the distal end surface of the endoscope can be disposed facing the surface of the heart while keeping an observation distance from the surface of the heart. Accordingly, a bird's eye view of the surface of the heart can be observed.

In the above aspect, the sheath body may have a distal end provided with a sheath bending section that is bendable toward the pressing surface, and the lifting part may be formed of the sheath bending section.

Accordingly, by adjusting the amount by which the protruding section is lifted from the surface of the heart in accordance with the bending angle of the sheath bending section, the distance between the distal end surface of the endoscope and the surface of the heart can be controlled.

In the above aspect, the sheath may include a bending wire extending from a distal end of the sheath bending section toward a base end in the longitudinal direction of the sheath body, and may also include a bend control section that is provided at the base end of the sheath body and that pulls the base end of the bending wire so as to cause the sheath bending section to bend. The bending wire may be disposed in the sheath bending section so as to be exposed to the outside.

Accordingly, since contact between the bending wire and other components at the outer side of the sheath bending section is reduced, the pulling force applied to the bending wire by the bend control section is efficiently transmitted to the distal end of the sheath bending section. Consequently, the sheath bending section can stably lift the protruding section and the pericardium with a larger force.

In the above aspect, the protruding section may have enough rigidity for maintaining a shape having a curvature smaller than a curvature in a natural state of the pericardium against the elastic force of the pericardium within the pericardial cavity, and the lifting part may be formed of the protruding section.

Accordingly, the protruding section can be disposed at a position away from the surface of the heart toward the pericardium within the pericardial cavity by utilizing the rigidity of the protruding section.

In the above aspect, the opening may be provided at a distal end of the protruding section.

Accordingly, the protruding section can be reduced in size in the longitudinal direction.

In the above aspect, the opening may be provided at an intermediate position of the protruding section in the longitudinal direction.

Accordingly, by means of the distal end of the protruding section extending toward the distal end of the opening, a space between the heart and the pericardium can be ensured at the distal end of the opening, so that a wider region of the surface of the heart can be observed.

In the above aspect, the sheath may include a transparent plate that is provided at a distal end of the protruding section so as to intersect with the longitudinal direction and that is transparent with respect to visible light.

Accordingly, in a state where the endoscope bending section is extended in the longitudinal direction of the sheath body within the protruding section, the visual field in front of the sheath can be observed through the transparent plate. Moreover, in a case where the opening is provided at the distal end of the protruding section, the endoscope is inserted into the sheath to a position where the distal end surface of the endoscope abuts on the transparent plate, so that the endoscope bending section can be disposed at the same position as the opening in the longitudinal direction.

In the above aspect, the sheath may include an illuminating section that is provided near the opening and that outputs illumination light outward in the radial direction of the opening.

Accordingly, the visual field of the endoscope can be illuminated by using the illuminating section provided in the sheath, so that an illuminating component can be eliminated from the endoscope, whereby the endoscope can be reduced in diameter.

A second aspect of the present invention is directed to a pericardial endoscope system comprising: the pericardial endoscope sheath according to claim 1; and an endoscope that is inserted into the pericardial endoscope sheath in a movable manner in a longitudinal direction and that is provided with an endoscope bending section at a distal end.

In the above aspect, the endoscope may have a marker indicating that an amount by which the endoscope is inserted in the sheath is equal to an amount by which the endoscope bending section is disposed at the same position as the opening in the longitudinal direction.

Accordingly, based on the marker, the operator can easily recognize that the endoscope has been inserted into the sheath to a position where bird's-eye-view observation is possible.

In the above aspect, the endoscope may be of a direct viewing type or an oblique viewing type.

Accordingly, especially in the case where the endoscope is of an oblique viewing type, the bending angle of the endoscope bending section can be reduced when performing bird's-eye-view observation of the surface of the heart, so that a larger observation distance can be ensured, whereby a wider region of the surface of the heart can be observed at one time.

The present invention is advantageous in that a bird's eye view of the surface of the heart can be observed.

### REFERENCE SIGNS LIST

1 sheath
2 endoscope
2a distal end surface
4 transparent plate
5 illuminating section
11 sheath body
12 operating section
12a bend control section
13 protruding section (lifting part)
14, 141, 142 sheath bending section (lifting part)
15 opening
16 pressing surface
17 bending wire
21 endoscope bending section
22 insertion section
23 operating section
23a bend control section
31, 32, 33, 34, 35 marker
50 dilator
51 guide wire
60 sheath
100 pericardial endoscope system

1. A pericardial endoscope sheath that is narrow and cylindrical and that is insertable into a pericardial cavity between a heart and a pericardium, the pericardial endoscope sheath comprising:
a flexible cylindrical sheath body that is bendable in conformity to a shape of a surface of the heart within the pericardial cavity and that has openings at opposite ends,
a protruding section that protrudes in a longitudinal direction of the sheath body from a ring-shaped distal end surface of the sheath body, and
a lifting part that generates a force for lifting the protruding section disposed within the pericardial cavity to a position separated from the surface of the heart toward the pericardium against an elastic force of the pericardium,
wherein the protruding section includes
a pressing surface that is provided in a circumferential portion centered on a longitudinal axis of the sheath body and that is disposed toward the pericardium when the protruding section is lifted by the lifting part so as to press against the pericardium, and
an opening that is provided at a position facing the pressing surface in a radial direction and at which a distal end surface of the endoscope protruding to the protruding section from the opening in the distal end surface of the sheath body is capable of appearing and disappearing in the radial direction as the endoscope bending section operates, in a state in which the endoscope is inserted into the sheath in a movable manner in a longitudinal direction.

2. The pericardial endoscope sheath according to claim 1,
wherein the sheath body has a distal end provided with a sheath bending section that is bendable toward the pressing surface, and
wherein the lifting part is formed of the sheath bending section.

3. The pericardial endoscope sheath according to claim 2, further comprising:
a bending wire extending from a distal end of the sheath bending section toward a base end in the longitudinal direction of the sheath body; and
a bend control section that is provided at the base end of the sheath body and that pulls the base end of the bending wire so as to cause the sheath bending section to bend,
wherein the bending wire is disposed in the sheath bending section so as to be exposed to the outside.

4. The pericardial endoscope sheath according to claim 1,
wherein the protruding section has enough rigidity for maintaining a shape having a curvature smaller than a curvature in a natural state of the pericardium against the elastic force of the pericardium within the pericardial cavity, and
wherein the lifting part is formed of the protruding section.

5. The pericardial endoscope sheath according to claim 1,
wherein the opening is provided at a distal end of the protruding section.

6. The pericardial endoscope sheath according to claim 1,
wherein the opening is provided at an intermediate position of the protruding section in the longitudinal direction.

7. The pericardial endoscope sheath according to claim 1, further comprising:
a transparent plate that is provided at a distal end of the protruding section so as to intersect with the longitudinal direction and that is transparent with respect to visible light.

8. The pericardial endoscope system according to claim 1, further comprising:
an illuminating section that is provided near the opening and that outputs illumination light outward in the radial direction of the opening.

9. A pericardial endoscope system comprising:
the pericardial endoscope sheath according to claim 1; and
an endoscope that is inserted into the pericardial endoscope sheath in a movable manner in a longitudinal direction and that is provided with an endoscope bending section at a distal end.

10. The pericardial endoscope system according to claim 9,
wherein the endoscope has a marker indicating that an amount by which the endoscope is inserted in the pericardial endoscope sheath is equal to an amount by which the endoscope bending section is disposed at the same position as the opening in the longitudinal direction.

11. The pericardial endoscope system according to claim 9,
wherein the endoscope is of a direct viewing type or an oblique viewing type.
